(19) United States
(12) Patent Application Publication
(10) Pub. No.: US 2017/0128503 A1
(43) Pub. Date: May 11, 2017

(54) NEURO-MODULATING PREPARATIONS FOR TREATMENT OF NEUROLOGICAL AND MOOD DISORDERS

(71) Applicants: Stefano SCOGLIO, Urbino Pu (IT); Franco CANESTRARI, Urbino Pu (IT); Serena BENEDETTI, Urbino Pu (IT); Yanina BENEDETTI, Urbino Pu (IT); Maria DELGADO-ESTEBAN, Salamanca (ES)

(72) Inventors: Stefano SCOGLIO, Urbino Pu (IT); Franco CANESTRARI, Urbino Pu (IT); Serena BENEDETTI, Urbino Pu (IT); Yanina BENEDETTI, Urbino Pu (IT); Maria DELGADO-ESTEBAN, Salamanca (ES)

(21) Appl. No.: 15/377,315

(22) Filed: Dec. 13, 2016

Related U.S. Application Data
(63) Continuation-in-part of application No. 12/306,483, filed on Aug. 26, 2009, now Pat. No. 9,522,167.
(60) Provisional application No. 60/816,593, filed on Jun. 27, 2006.

(30) Foreign Application Priority Data
Jun. 26, 2007 (EP) PCT/EP2007/005622

(51) Int. Cl. A61K 35/748; A61K 31/198; A61K 31/137; A61K 38/16

(52) U.S. Cl. CPC A61K 35/748; A61K 38/164; A61K 31/198; A61K 31/137

(57) ABSTRACT

Disclosed are compositions of matter, which are extracts of the microalga Aphanizomenon flos Aquae Aquae Rafts ex Born. Rah. Var. flos aquae (AFA-Klamath), and purified components thereof. These compositions are useful for the treatment of neurological and neurodegenerative diseases, and of mood conditions. These diseases and conditions include conditions and disorders such as Alzheimer's disease, Parkinson's disease, multiple sclerosis, hyperactivity and attention deficit disorders, autism, depression, memory deficit, and mood disturbances.

### CONTINUITY DATA

The present application is a Continuation-in-Part application and claims the benefit of prior filed U.S. application Ser. No. 15/348,667 filed 10 Nov. 2016 & Ser. No. 12/306,483 filed 26 Jun. 2007, and International Application serial number PCT/EP2007/005622 filed 26 Jun. 2007; which in turn claim priority to U.S. provisional application Ser. No. 60/816,593 filed 27 Jun. 2006, and all said prior applications being incorporated herein by reference in their entirety.

### FIELD OF THE INVENTION

The present invention is in the field of bio-affecting/body treating compositions (Class 424). Specifically, the present invention relates to extracts and materials containing or obtained from an alga as an active ingredient (subclass 195.17). More specifically, the present invention relates to extracts of the microalga Aphanizomenon Flos Aquae (AFA-Klamath), and purified components thereof.

### BACKGROUND OF THE INVENTION

Phenylethylamine (“PEA”) is an endogenous amine synthesized by decarboxylation of phenylalanine in dopaminergic neurons of the nigrostriatal system. PEA is believed to act as a neuromodulator of neurotransmissions in the brain by promoting the neurotransmission of catecholamines. It is known that PEA stimulates the release of acetylcholine as well as dopamine. Furthermore PEA increases norepinephrine (NE) neurotransmission and even serotonin neurotransmission. Recently it has been shown that PEA can also work as an autonomous neurotransmitter, with its specific neuronal receptors; and that it acts as a true neuromodulator, being also able to depress neurotransmission. From this derive a whole series of effects: stimulation of attention and memory; mood enhancement, with significant antidepressant activity; promotion of empathy and thus sociality, included emotional and sexual behavior; inhibition of hunger; reduction of the need for substance abuse and drug dependency.

The link between PEA and emotional mood has been confirmed by studies whereby significantly lower levels of PEA, measured as such or through its metabolite PAA (phenylacetic acid) in the plasma or urines, have been found in depressed subjects. Also, it has been seen that Parkinson's patients have significantly lower levels of PEA, as measured directly in the plasma. The progressive reduction of neurotransmission, particularly dopaminergic, in these patients, is related to the progressive degeneration of the dopaminergic neurons of the substantia nigra.

This reduction in the PEA levels goes together with a parallel increase in levels of MAO-B in Parkinson patients, hence the drugs used in Parkinson's are MAO-B inhibitors such as selegiline. Moreover, once ingested PEA can easily pass through the blood-brain barrier and stimulate the release of dopamine from the nigrostriatal tissue even at low dosages. This is an important distinctive character, because the drug currently used, selegiline, while inhibiting MAO-B and the reuptake of dopamine, does not have any action on its release from the nigrostriatal tissue, and so it does not help to produce more dopamine, a serious limit in a pathology such as Parkinson, where the very generation of dopamine is greatly jeopardized.

Alzheimer's disease is a neurodegenerative disorder involving the mechanism of production and reuptake of dopamine and the progressive destruction of the neurons of the striatal area, which over time brings to a low number of dopaminergic neurons, and consequently of dopamine transmission. Although there are no clear data on the fact that ADHD (Attention Deficit Hyperactivity Disorder) entails a neurodegenerative pathology, some studies have tried to prove that neuronal degeneration is a cause of ADHD in both children and adults. Most importantly there are evidences whereby the children affected by ADHD and learning disabilities have significantly lower levels of PEA, and so a reduction in the neuro-modulation of attention (dopamine) and sedation (serotonine). That is why the drug of choice for ADHD is methylphenidate, a synthetic derivative of PEA, which also acts by stimulating a higher production of PEA, and thus of dopamine and norepinephrine, two neurotransmitters directly involved in the etiology of ADHD.

The use of amphetamines to control hunger and, consequently, weight is well known. Their use in this area has always been controversial due to their side effects, which tend to become potentially very serious over time. This is confirmed by the fact that the main drugs currently used for hunger and weight control are amphetamine-like dopaminergic antidepressants, such as venlafaxine and buproprion. These molecules, as all amphetamines, are synthetic derivatives of PEA. The latter acts as a potent appetite suppressant insofar as its degradation by MAO-B enzymes is prevented.

Monoaminoxidase (MAO) A and B catalyze the degradation of neuroactive and vasoactive amines in the CNS and in peripheral tissues, including PEAs. MAO-B in particular, given its direct and indirect relevance to dopaminergic transmission, is involved in neurological disorders where dopamine is essential, such a depression and mood disorders, Parkinson and Alzheimer diseases. For this reason, MAO-B inhibitors are used in the treatment of such neurological disorders.

### SUMMARY OF THE INVENTION

The invention is based on constituent properties of the microalga Aphanizomenon Flos Aquae Aquae Ralfs ex Born. & Flah. Var. flos aquae (herein “AFA-Klamath”). These constituents, alone or in combination, can exert beneficial effects on various neurological diseases, conditions, dysfunctions or disorders, including neurodegenerative diseases such as Alzheimer's and Parkinson's, multiple sclerosis, hyperactivity and attention deficit disorders (ADHD), autism, depression, memory deficit and mood disturbances. In particular, it has been found that the AFA-Klamath microalga contains phenylethylamine (PEA), which is a neuro-modulator characterized by dopaminergic and noradrenergic activity, and other specific molecules, which quite surprisingly have proved to be very effective inhibitors of the enzyme monoaminoxidase B (MAO-B). These are: a) the specific AFA-phytochrome; b) the AFA-phycobiliprotein complex containing a phycobilisome formed by C-phycocyanin (C-PC), and phycoerythrocyanin (“PEC”), including its chromophore phycoviolobilin (or “PVB”)) (herein called: “AFA-phycocyanin(s)”); and c) mycosporine-like amino acids (“MAAs”). This result is very important since the PEA contained in the algae, unless protected by the constituent MAO-B inhibitors, would be rapidly destroyed upon ingestion by the MAO-B enzyme. The same molecules that act as MAO-B selective inhibitors, also perform a powerful neuro-protective role, thus significantly enhancing the ability of the extract to promote neurological health.

Accordingly the present invention provides a method for preventing, controlling or treating the above mentioned neurological diseases, conditions, dysfunctions or disorders by administering to a subject in need thereof an AFA-Klamath preparation, particularly an extract enriched in such active components, or an isolated and purified component selected from: a) the AFA phytochrome, b) the C-phycocyanin/phycoerythrocyanins complex, as present in AFA-Klamath or in any other microalgae; c) the mycosporine-like amino acids Porphyra and Shinorine, as present in AFA-Klamath or from any other algal source; d) or a mixture thereof.

Preferably the AFA-Klamath extract according to the present invention is prepared by the following steps: a) freezing the freshly harvested AFA-Klamath alga and thawing it, or, if the starting material is dried AFA-Klamath powder, then sonicating the water-diluted AFA-Klamath powder to disrupt the cells; b) centrifuging the product of step a) to separate the supernatant (retaining most of the cytoplasmic portion) from the precipitate (retaining most of the cell wall fraction); c) collecting the supernatant containing the water-soluble components. The resulting product is an extract (indicated as “Basic Extract”), which concentrates PEA as well as other synergic molecules such as the AFA-phytochrome, the AFA-phycocyanins, and the MAAs. For example, whereas AFA-Klamath microalga has a natural content of PEA ranging from 2 to 4 mg/gr, the Basic Extract increases this concentration to a level ranging from 9 to 11 mg/gr (HPLC analysis).

It is possible to further purify the extract by passing it through an ultra-filtration system, preferably through a membrane with a molecular weight cut-off of 30,000 Daltons. The ultra-filtration retentate (Extract A) contains as major active components both the AFA-phycocyanins (mol. weight=121,000) and the AFA-phytochrome (mol. Weight=480,000). Interestingly, even though MAAs have a molecular weight well below the cut-off size employed, the retentate also increases the concentration of MAAs.

The Basic Extract obtained by steps a) to c), i.e. without ultra-filtration, is generally preferred as it contains the most appropriate amounts of PEA, AFA-phytochrome, AFA-PC and MAAs. Moreover, this Basic Extract also includes substances such as chlorophyll and carotenes, even though in a reduced proportion, contributing to its antioxidant and anti-inflammatory properties. As an alternative, the active components of AFA-Klamath, namely the complex C-phycocyanin/phycoerythrocyanin (C-PC/PEC), AFA-phytochrome and MAAs can be isolated and purified, as further described below, and used in a method according to the invention.

In a preferred embodiment, AFA-Klamath C-PC/PEC complex, AFA-phytochromes and mycosporine-like amino acids are used as a combined preparation for simultaneous or separate administration to a subject in need thereof; in a yet further preferred embodiment, such a combined preparation contains phenylethylamine as an additional active ingredient. Among the mycosporine-like amino acids, Shinorine and Porphyra-334 are particularly preferred, as they are contained in relatively higher concentration in AFA-Klamath microalgae. The observed inhibition of monoaminoxidase-B is particularly relevant, as it promotes increase dopaminergic transmission and minimizes the catabolism of PEA. Significantly, both phytochrome and AFA-phycocyanin inhibit MAO-B in a reversible and mixed way, whereas MAO-B inhibition by MAAs is competitive and reversible; therefore, all three molecules assure high efficacy in physiological conditions with the absence of side effects.

In a further aspect, the invention is directed to a nutraceutical, that is, a pharmaceutical-grade and standardized nutrient or nutritional supplement comprised of a preparation, extract, or isolated component of AFA-Klamath. The preferred components being selected from: the C-PC/PEC complex, as present in AFA-Klamath or from any other microalgal source, or the isolated C-PC and PEC single components; the AFA-phytochrome; the mycosporine-like amino acids Porphyra and Shinorine, as present in AFA algae or from any other algal source; and/or a mixture thereof. Additionally, phenylethylamine PEC is includable. In a preferred embodiment, the nutritional compositions are dietary supplements in the form of tablets, capsules, liquids, etc. In a further preferred embodiment the pharmaceutical compositions can be provided in the form of tablets, capsules, sachets, syrups, suppositories, vials and ointments and can be used for the prevention or treatment of neurological or neurodegenerative diseases or conditions indicated above. The AFA-Klamath liquid extracts according to the invention can be used as such or can be dried through methodologies such as freeze-drying, spray-drying or the like. The isolated active components can be formulated using techniques and following procedures that are known to anyone skilled in the art.

The dose of active ingredient will depend on the intended use of the compositions, whether as nutritional supplement or as a pharmaceutical preparation. The effective amount of each component will be generally comprised in the following ranges: PEA=0.1-100 mg, preferably 5-30 mg; phytochrome=0.1-1,000 mg, preferably 0.8-10 mg; MAAs=0.1-1,000 mg, preferably 10-100; and phycocyanins=1-2,500 mg, preferably 50-1,000 mg.

### BRIEF DESCRIPTION OF THE DRAWINGS

FIG. 1 shows the absorption of partially purified mycosporine-like amino acids (MAAs) from an AFA-Klamath sample.

FIG. 2 shows chromatograms from partially purified AFA-Klamath samples.

FIG. 3 shows UV spectra of purified MAAs.

FIG. 4 shows a comparison of components of a cellular lysate of AFA-Klamath with those of a Synechocystis PCC 6803 cyanobacterium.

FIG. 5 shows the absorption of a crude extract of AFA-Klamath and a purified sample of AFA-PC.

FIG. 6 shows the absorption of a purified AFA-PCB chromophore.

FIG. 7A compares the MAO-B activity of a water-soluble fraction and a lipophilic fraction of an AFA-Klamath extract.

FIG. 7B compares the MAO-A and MAO-B activity of a water-soluble fraction of an AFA-Klamath extract.

FIG. 8 shows a Lineweaver-Burk plot of a water-soluble fraction of an AFA-Klamath extract.

FIG. 9 shows the MAO-B activity of a purified AFA-PC sample.

FIG. 10 shows a Lineweaver-Burk plot of a purified AFA-PC sample.

FIG. 11 shows the MAO-A and MAO-B activity of MAAs from an AFA-Klamath sample.

FIG. 12 shows a Lineweaver-Burk plot of MAAs from an AFA-Klamath sample.

FIG. 13 shows a graph of slope versus concentration of MAAs from an AFA-Klamath sample.

FIG. 14 shows the MAO-B inhibitory activity of the three molecules of AFA-Klamath.

FIG. 15 shows the dose-dependent MAO-B activity of an AFA-phytochrome.

FIG. 16 shows a Lineweaver-Burk plot of an AFA-phytochrome.

FIG. 17 shows the MAO-B activity of an AFA-phytochrome.

FIG. 18 shows the % apoptosis of an AFA-Klamath extract with added glutamate.

FIG. 19 shows the % apoptosis of MAAs from an AFA-Klamath extract with added glutamate.

FIG. 20 shows the % apoptosis of phytochrome (“PC”) from an AFA-Klamath extract with added glutamate.

FIG. 21 shows the % apoptosis of chromophore phycocyanobilin (PCB) from an AFA-Klamath extract with added glutamate.

FIG. 22 shows the relation of the AFA-phytochrome band and the phycobilisome bands and gives an indication of the molecular weight of the AFA-phytochrome present in algae.

FIG. 23 shows the light absorption properties of the AFA-phytochrome at 672 nm and 694 nm, which corresponds respectively to red-absorbing and far-red absorbing forms in a state of equilibrium.

FIG. 24 shows that % MDA formation of AFA-phytochrome after incubation with plasma.

FIG. 25 is a schematic representation of the MAAs (“mycosporine-like amino acids”) Porphyra-334 and Shinorine.

### DETAILED DESCRIPTION OF THE INVENTION

Identification of “AFA-Phytochrome”, a Unique Phytochrome Typical of Klamath Algae:

Phytochromes are photoreceptors, pigments that plants use to detect light and that are sensitive to light in the red and far-red region of the visible spectrum. They perform many different functions in plants, including the regulation of flowering (through circadian rhythms), germination and the synthesis of chlorophyll. The latter is particularly relevant in relation to AFA algae, because the presence of this unique type of phytochrome in AFA-Klamath may be explained by its lack of the other phycobiliprotein commonly used by other cyanobacteria to complement C-phycocyanin in the process of photosynthesis, namely allo-phycocyanin. While the place of allo-phycocyanin in Klamath algae is taken by phycoerythrocyanin or PEC (see below), it is likely that PEC alone is not sufficient, especially considering that Klamath algae lives in a non-tropical environment which needs a high light harvesting efficiency, and so AFA algae seem to integrate their higher needs with the phytochrome.

The AFA-phytochrome, which has a peculiar structure, is described here for the first time. Over the years, different types of phytochromes have been found in plants, which not only have different phytochrome genes (3 in rice and 6 in maize, for instance), but in most cases they have significantly different protein components and structure. What makes them all phycochromes is that they all use the same biliprotein, called phytochromobilin, as a light-absorbing chromophore. This chromophore is similar to the phycocyanin's chromophore phycocyanobilin, and is characterized by a single bilin molecule consisting of an open chain of four pyrrole rings (tetrapyrroles). More specifically, in its Pᵣ normal state this biliprotein absorbs light at a maximum of 650-670 nm, whereas when activated by red light it is transformed into P_{fr} with an absorbance maximum of 730 nm.

The first cyanobacterial phytochrome to be discovered (Synechocystis) showed to have a weak structural similarity with plant phytochromes. Nevertheless, Synechocystis' biliprotein is generally considered a phytochrome insofar as it is a red/far-red reversible chromoprotein.

AFA Phytochrome Purification and Characterization:

AFA-phytochrome has a biliprotein as its chromophore that absorbs light in the red/far-red spectrum. To establish its structure and activities we have purified the phytochrome with the following protocol:

Suspend 1 g of extract in 10 ml of 1 K-phosphate buffer, pH 7.0.

Vortex twice for 1 min with half their volume.

Incubate cells for 35′ with 2% Triton X 100.

Centrifuge at 28,000 rpm for 16-18 hrs.

Collect supernatant on a sucrose density step gradient.

Spin the gradient using swing-out rotors at 150,000×g for 12 hrs, and store at −20° C.

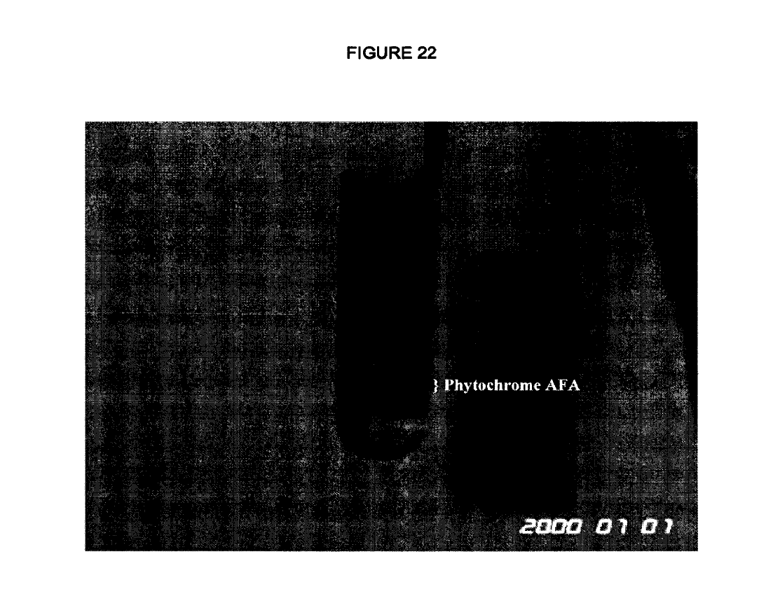

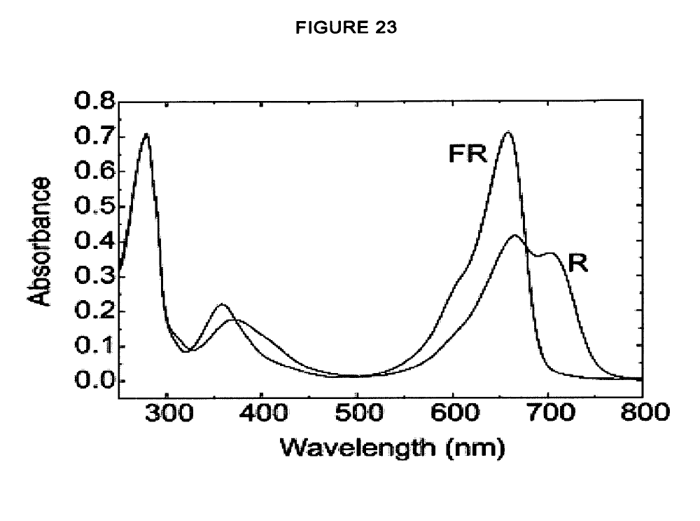

The phytochrome corresponds to the lysate band of an intense orange color, which is visible at approximately 1M of sucrose, while the phycobilisome stands at approximately 0.75 M. This relation of the two bands also gives a reliable indication about the molecular weight of the phytochrome present in the algae, which is about 4 times that of the trimeric AFA-PC: the latter being 121 Kd, we can preliminarily establish the MW of AFA-phytochrome at approximately 480 Kd (FIG. 22). When tested for its light-absorbing properties, the phytochrome shows to absorb light with two peaks at 672 nm and 694 nm, which corresponds respectively to Pᵣ (red-light absorbing) e P_{fr} (far-red light absorbing) forms in a state of equilibrium (FIG. 23).

As to the quantity of phytochrome contained in AFA-Klamath, our first evaluation gives the following preliminary result: 2 mg/gr (or 0.2% Dry Weight (“DW”)). As to the extracts, the concentration increases to approximately 0.5% in the Basic Extract, and approx. 1% in the Extract B. These are low concentrations, yet the antioxidant/anti-inflammatory potency of this molecule is so strong that even a very small quantity can produce very relevant effects.

Antioxidant Activity:

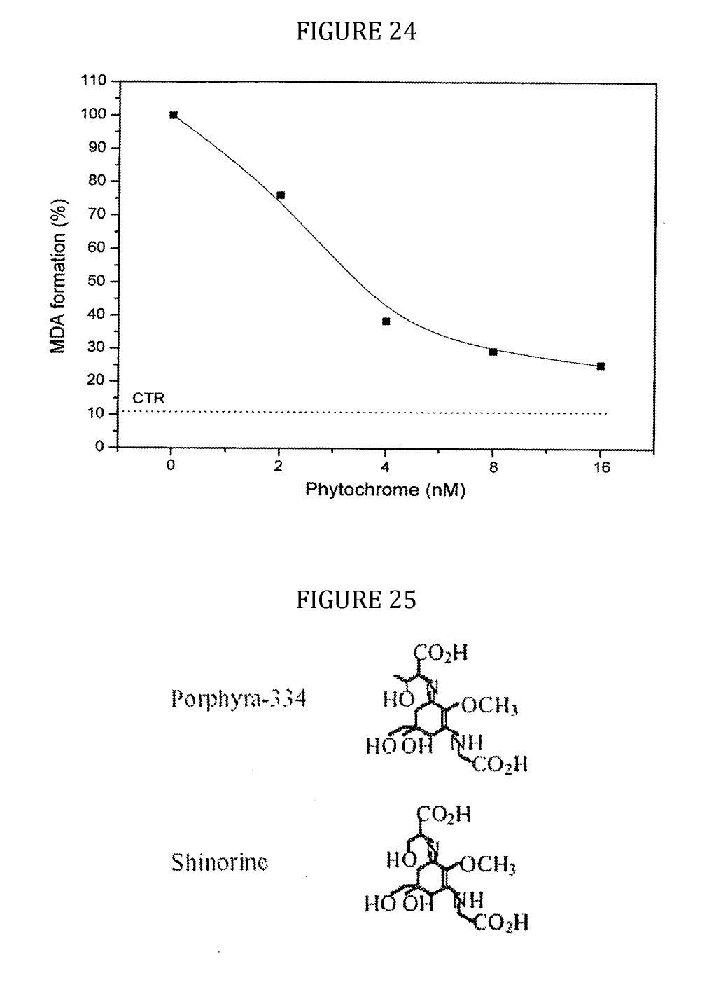

The purified AFA-phytochrome has shown to be a very powerful antioxidant. In fact, in absolute terms it is the most powerful molecule so far found in Klamath algae. The incubation for 2 hrs of human plasma samples with oxidative agent CuCl₂ at 100 μM generates increased levels of malondialdehyde (MDA), a late byproduct of lipid peroxidation, which is measured through spectrophotometer at 535 nm after a reaction with thiobarbituric acid (TBA test). When plasma is incubated for 2 hrs at 37° C. with CuCl₂ 100 μM together with increasing quantities of AFA-phytochrome (2-16 nanomolar (“nM”)) extracted from AFA algae, a very strong dose-dependent reduction of the MDA levels is observed (FIG. 24). In fact, an almost complete inhibition of lipoperoxidation is obtained with MDA levels close to control, with just 16 nM of AFA-phytochrome. Significantly, the IC50 of 3.6 nM is 45 times less than that obtained for the PCB. The phytochrome is the main responsible for the antioxidant and neuroprotective effect of the Basic Extract, which are higher than those of AFA-PC.

Extraction, Purification and Quantification of MAAs:

We tested the presence of MAAs in the cyanophyta Aphanizomenon flos-aquae of Klamath Lake, generally known as Klamath algae. To our knowledge, only a very recent report exist on the occurrence of MAAs in any Aphanizomenon species; however, such report only identifies porphyra as the MAAs present, whereas our research shows the presence of two MAAs, both Porphyra and Shinorine (see FIG. 25). On the other hand, in relation to the overall literature on algae, whereas most of the cyanobacteria reported to date contain Shinorine as their primary MAAs, we found a rare occurrence of porphyra-334 as the primary MAA in Aphanizomenon flos-aquae in addition to Shinorine.

MAAs were extracted as previously reported. Briefly, 20 mg of AFA-Klamath powder or 20 mg. of aqueous extract are extracted in 2 ml of 20% (v/v) aqueous methanol (HPLC grade) by incubating in a water bath at 45° C. for 2.5 hrs. After centrifugation (@ 5,000×g; GS-15R Centrifuge, Beckman, Palo Alto, USA), the supernatant was evaporated to dryness and re-dissolved in 2 ml 100% methanol, then vortexed for 2-3 min and centrifuged at 10,000 G for 10 min. The supernatant was evaporated and the extract re-dissolved in the same volume of 0.2% acetic acid for the analysis in HPLC or in 200 μl of phosphate buffer (PBS) for the evaluation of antioxidant properties. The samples were filtered through 0.2 μm pore-sized syringe filters (VWR International, Milan, Italy) before being subjected to HPLC analysis, or to the test of antioxidant properties (see below).

The MAAs of the Klamath algae have an absorption maximum of 334 nm. Further purification of MAAs was done using a HPLC system (Jasco Corporation, Tokyo, Japan) equipped with an Alltima C18 column and guard (4.6×250 mm i.d., 5 μm packing, Alltech, Milan, Italy), according to the literature. The wavelength for detection was 330 nm; the mobile phase was 0.2% acetic acid at a flow-rate of 1.0 ml/min. Identification of MAAs was done by comparing the absorption spectra and retentions time with standards such as Porphyra and Pterocladia sp., mainly containing porphyra-334, Shinorine and palythine, kindly provided by Dr. Manfred Klisch, Friedrich-Alexander-Universitat, Erlangen, Germany. Absorption spectra of samples were measured from 200 to 800 nm in a single-beam spectrophotometer (DU 640, Beckman, Palo Alto, USA). The raw spectra were transferred to a computer and treated mathematically for the peak analyses of MAAs.

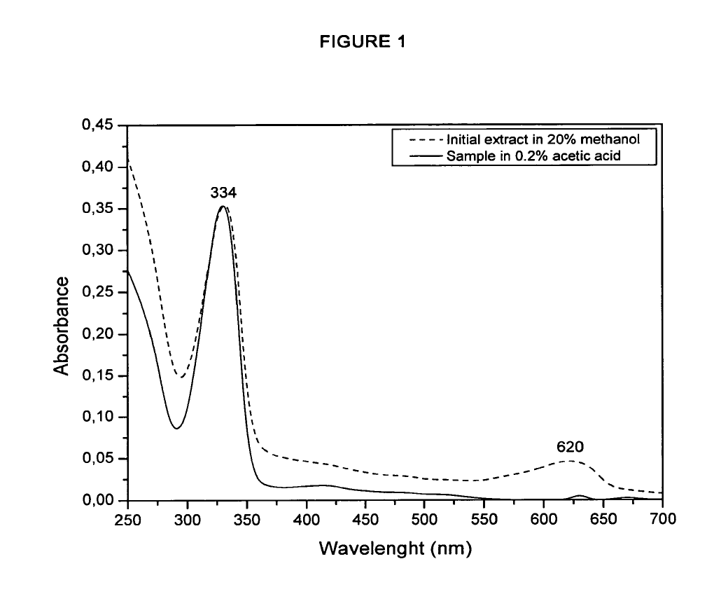

MAAs were partially purified from AFA-Klamath sample and from the aqueous extract as described earlier. Extraction of samples with 20% methanol at 45° C. for 2.5 hrs resulted in a prominent peak at 334 nm (MAAs); even if small amounts of photosynthetic pigments (such as phycocyanin at 620 nm) were also extracted with this procedure (see FIG. 1, dashed line). MAA samples were further treated with 100% methanol in order to remove proteins and salts and finally with 0.2% acetic acid to remove non polar-photosynthetic pigments. The resultant partially purified MAAs had an absorption maximum at 334 nm (FIG. 1, solid line).

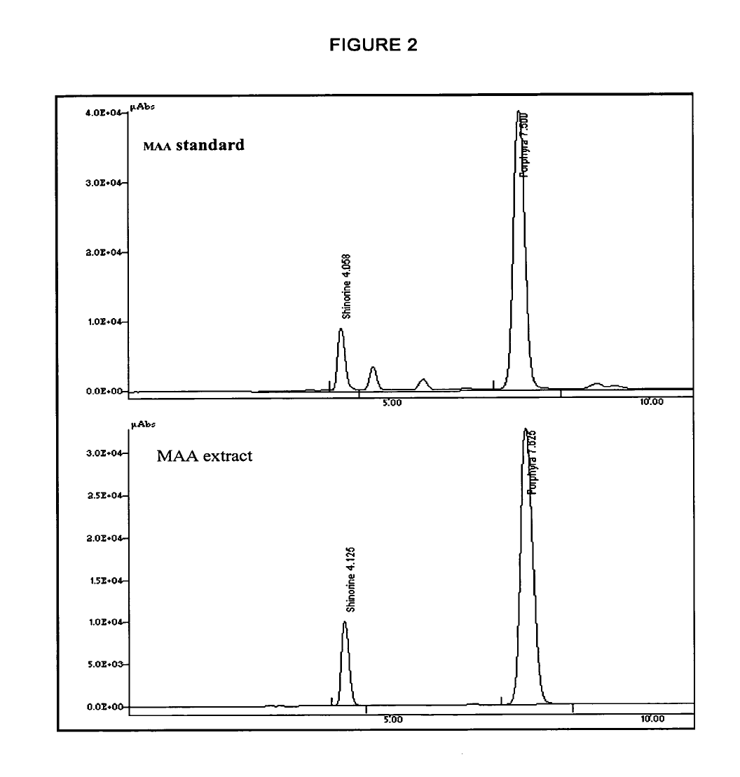

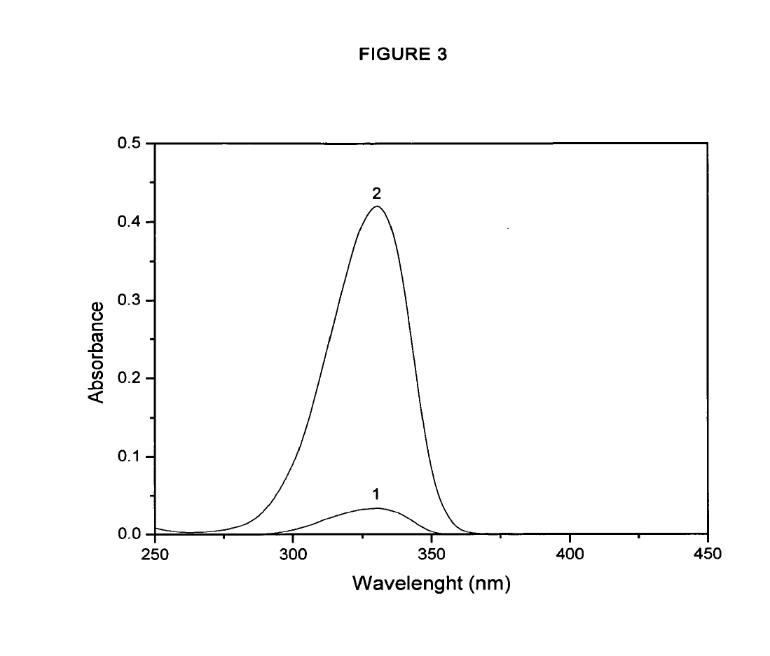

Further analysis and purification of MAAs was done by HPLC with a view to find whether the compounds absorbing at 334 nm was a single MAA or a mixture of more than one MAAs. The chromatogram of the sample (FIG. 2) shows the presence of two MAAs with retention times of 4.2 (peak 1) and 7.6 min (peak 2) that were identified as shinorine and porphyra-334, respectively. Porphyra-334 seems to be the major MAA in AFA-Klamath since shinorine was present only in small quantities (peak area ratio 1:15). The UV spectra of the purified MAAs confirmed their absorption maximum at 334 nm (FIG. 3).

Taking into account that the molar extinction coefficients at 334 nm for Shinorine and porphyra-334 are of 44,700 and 42,300 M⁻¹cm⁻¹, respectively, we calculated: a) for AFA algae, concentrations of 0.49 mg g⁻¹ DW for Shinorine and 7.09 mg g⁻¹ DW for Porphyra-334; the total MAAs content being thus equal to 0.76% algal DW; b). For the Basic Extract, concentrations of 17-21 mg of MAAs (that is 1.7-2.1% DW). These data are significant, as the whole AFA-Klamath contains high constitutive levels of MAAs (0.76% DW), close to the maximal concentration found under UV exposure, i.e. 0.84%. Also, we found that the extract has a higher concentration than the whole algae, reaching levels that are much higher than the maximal potential concentration. The MAAs Shinorine and Porphyra-334 are structurally simple molecules (see FIG. 25), with a molecular weight of 300. This allows these water-soluble molecules to easily cross the blood-brain barrier, confirming their ability to express their MAO-B inhibitory potential in the area where it is mostly needed—the brain.

Phycocyanin (“PC”):

The phycocyanins are present in the extract at a concentration of 8-10% (for the quantification, see below). Phycocyanins are the blue pigments typical of all cyanobacteria or blue-green algae, although with peculiar characteristics for each specific microalga. As to functional and therapeutic properties of phycocyanins, research has mostly focused so far on those of the microalga Spirulina. The purified phycocyanins from Spirulina have been shown to possess antioxidant and anti-inflammatory properties on different physiological systems such as liver, respiratory system and brain. Such properties of the purified PC from Spirulina can in general be attributed also to the phycocyanins of other algae, given their substantial similarity. Nevertheless, there can exist species-specific differences in the different phycocyanins from different microalgae, which can lead to a different potency in the explication of the above-described functional and therapeutic properties.

Structure and Specific Characteristics of the Klamath Algae's Phycobilisomes:

Generally speaking, in the intact cyanobacterial cell phycocyanins (“PC”) are present inside the phycobilisome in the functional form (αβ)₆. Following the break-up of the cell, the protein can be found in different aggregation states (monomers, dimers, trimers, hexamers) according to the organism analyzed. In the case of Klamath algae, the electrophoretic analysis of the PC, both as contained in the extract and as purified from the extract itself, has shown that the protein is found for the most part in its trimeric form (αβ)₃, with a total molecular weight of 121,000. A monomer (αβ) has a molecular weigh of approximately 40,000 (18,500 for subunit α+21,900 for subunit β). The majority of the studies on the purified PC from Spirulina tell us instead that the protein is found in Spirulina in the monomeric form (αβ) with a molecular weight of approximately 37,500, thus showing a different aggregation state relative to the purified PC from AFA-Klamath. Chromatographic analysis of the AFA-phycobilisomes has also shown that, as in other cyanobacterial species, the α subunit of PC binds a single prosthetic group, while the β subunit binds two. The prosthetic group or chromophore is called phycocyanobilin (“PCB”) and is responsible both of the blue color of the protein and of its antioxidant power.

A fundamental difference between AFA-Klamath and Spirulina rests on the different structure of the phycobilisome. As opposed to Spirulina, the phycobilisome of AFA-Klamath does not contain the pigment allo-phycocyanin, but only the pigment C-phycocyanin bound to a structural component, which is missing in Spirulina, namely phycoerythrocyanin (“PEC”). PEC is a photosynthetic pigment, which currently has been identified only in a limited number of cyanobacterial species. PEC has a chemical structure very similar to that of PC, being composed of the two subunits α and β, which associate to form monomers and trimers. Nevertheless, while every monomer of PC binds 3 molecules of PCB, PEC possesses the unique characteristic of binding two molecules of PCB to the subunit β and one molecule of phycoviolobilin (PVB) to the α subunit, which is responsible of the purple color of the pigment. This absolutely is the first time that the phycobilisome of Klamath algae is defined as peculiarly constituted by the union of C-phycocyanin and phycoerythrocyanin, and this different qualitative structure of the phycobilisome of AFA-Klamath algae adds a further decisive factor distinguishing AFA-Klamath from Spirulina.

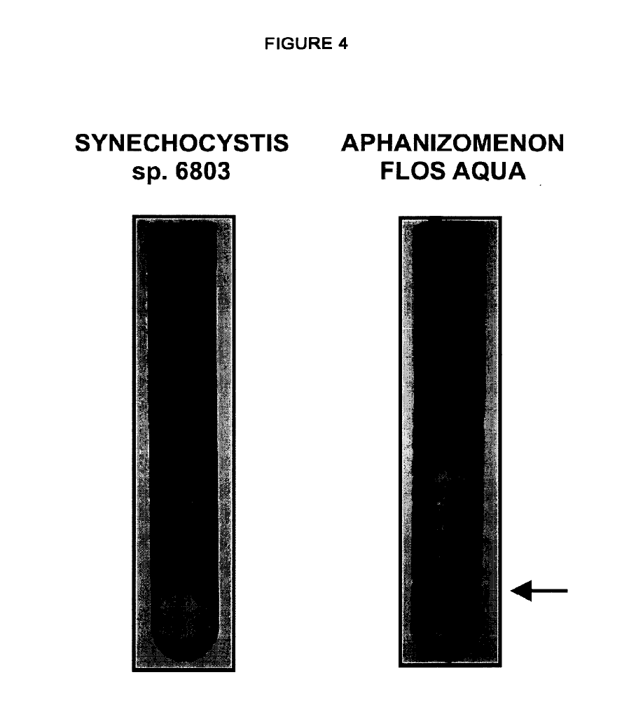

FIG. 4 confirms what has been said, comparing the components of the cellular lysate of AFA-Klamath with those of another well-known cyanobacterium: Synechocystis PCC 6803. In both cyanobacteria it possible to see the blue band representing the phycobilisome, but in AFA algae the phycobilisome presents a lower molecular mass, confirming that, as opposed to common microalgae such as Spirulina, in the AFA-phycobilisome only phycocyanins, but not allo-phycocyanins, are present. Furthermore, FIG. 4 shows that in AFA also present is a light purple band (shown by the arrow), which is typical of phycoerythrocyanins, thus proving their presence in the phycobilisome of Klamath algae.

To deepen the definition, each blue band has been further analyzed through HPLC connected to mass spectrometer (RP-HPLC-ESI-MS). Thanks to the different times of retention, the proteins of the phycobilisome have been separated and identified based on their molecular mass. The results obtained are shown in the following tables. First we see that while in Synechocystis (Table 1) both phycocyanin (cpcA at 28.2 min and cpcB at 28.9 min) and allo-phycocyanin (apcA at 30.7 min and apcB at 31.2 min), in AFA-Klamath (Table 2) only phycocyanin (cpcA at 28.8 min and cpcB at 30.0 min) is present. Secondly, in AFA-Klamath a protein with molecular mass of 19,469 has been identified which is not present in Synechocystis and which corresponds to the β-subunit of the phycoerythrocyanin with two bilins attached (pecB a 25.0 min).

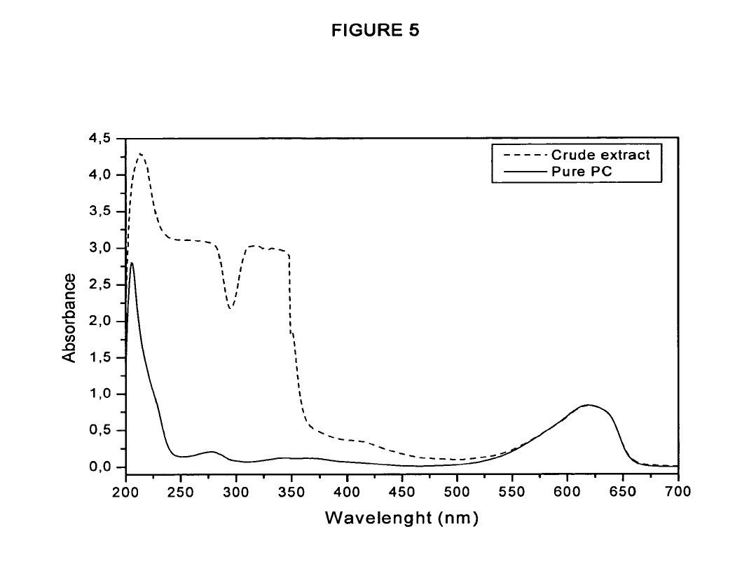

This unique structure is an important element to explain the stronger antioxidant and anti-inflammatory action of the whole AFA-PC relative to its PCB. Antioxidant and anti-inflammatory properties become relevant in this context insofar as they generate a strong neuroprotection; the whole PC is more powerful than its PCB also in terms of neuroprotection, which clearly indicates that the other active component besides PCB in the phycobilisome, namely PEC with its specific PVB chromophore, is very likely the most active health-enhancing principle in AFA-PC. That the purified AFA-PC does indeed contain not only the C-PC with its PCB chromophore, but also PEC and its PVB chromophore is evident by looking at the spectrometry of the extract resulting from the purification (FIG. 5).

TABLE 1
Proteins Present in the phycobilisome of Synechocystis

| Retention | Measured | Expected | Protein | NCBI |
|---|---|---|---|---|
| time | molecular | molecular | [homologous | Number |
| (min) | mass | mass | organism] | of access |
| 14.5 | 9322 | 9322 | cpcD | gi\|16329820 |
| 22.6 | 32505 | 32520 | CpcC | gi\|16329821 |
| | 32388 | 30797 | cpcC | gi\|16329822 |
| 24.6 | 28770 | 27392 | cpcG | gi\|16329710 |
| 24.8 | 28885 | 28522 | cpcG | gi\|16332194 |
| 28.2 | 18173 | 17586 | cpcA (sub α | gi\|2493297 |
| | | | phycocyanin) | |
| 28.9 | 19313 | 18126 | cpcB (sub β | gi\|2493300 |
| | | | phycocyanin) | |
| 30.7 | 17866 | 17280 | apcA (sub α | gi\|266765 |
| | | | allophycocyanin) | |
| 31.2 | 17816 | 17215 | apcB (sub β | gi\|266766 |
| | | | allophycocyanin) | |

In fact, the absorption maximum of C-PC is 620 nm, which in the spectrometry of FIG. 5 represents the top of the peak. But the absorption maximum of PEC is known to be 566 nm for the α-subunit (phycoviolobilin) and respectively 593 nm and 639 nm for the two PCBs of the β-subunit. All three values are indeed included in the bell-shaped peak constituting the spectrometric profile of the purified PC. In consideration of the strong link, very difficult to break, between C-PC and PEC in AFA algae, this confirms that besides the C-PC, also the PEC is necessarily part of the purified PC extract. This in turn means that the PC from AFA-Klamath is significantly different, both structurally and functionally, from the PCs of other cyanobacteria, including the one from Spirulina, on which most studies have been done; and that this difference consists in having only one part in common, namely C-PC, but not the other; with the consequence that, while the properties of C-PC can also be attributed to the C-PC component of the AFA-PC, the properties of the whole PC from AFA-Klamath, in its being a C-PC/PEC complex (including its chromophores PCB and PVB), are exclusively attributable to it (as well as to any C-PC/PEC complex present in any other microalgae).

Purification Methodologies:

PC was purified from the dried AFA-Klamath extract as follows:

suspend 500 mg of extract in 50 ml of 100 mM Na-phosphate buffer at pH 7.4;

centrifuge at 2,500 rpm for 10 min at 4° C.;

collect supernatant and add solid ammonium sulfate to a 50% saturation;

precipitate the proteins for 60 min at 4° C., while agitating sample;

centrifuge at 10,000 rpm for 30 min at 4° C.;

discard the clear/colorless supernatant and re-suspend the blue precipitate in a small volume of 5 mM Na-phosphate buffer pH 7.4;

dialyze overnight at 4° C. against the same buffer;

place the dialyzed PC in a hydroxyapatite column balanced with 5 mM Na-phosphate buffer at pH 7.4;

elute the sample with Na-phosphate buffer pH 7.0 of increasing ionic strength (from 5 to 150 mM);

collect the fractions and read the absorbance at 620 nm and 280 nm;

pool the fractions in which Abs.₆₂₀/Abs₂₈₀>4 (index of pure PC);

precipitate the PC with ammonium sulfate at 50% saturation for 1 hour at 4° C.;

centrifuge at 10,000 rpm for 30 min at 4° C.;

discard the supernatant and re-suspend the PC in 150 mM Na-phosphate buffer at pH 7.4;

dialyze against the same buffer at 4° C.;

transfer the purified PC to a flask and store in darkness between +4° C. to −20° C.

TABLE 2
Proteins Present in the Phycobilisome of AFA-Klamath Algae

| Retention | Measured | Expected | Protein | NCBI |
|---|---|---|---|---|
| time | molecular | molecular | [homologous | Number |
| (min) | mass | mass | organism] | of access |
| 15.2 | 9031 | 8925 | hypothetical protein | gi\|45510540 |
| | | | Avar03000795 | |
| | | | [Anabaena variabilis | |
| | | | ATCC 29413] | |
| | | 8895 | cpcD | gi\|131740 |
| | | | [Nostoc sp. PCC 7120] | |
| 25.0 | 19469 | 18284 | pecB: | gi\|548504 |
| | 19308 | | phycoerythrocyanin beta | |
| | | | chain | |
| | | | [Nostoc sp. PCC 7120] | |
| | | 18370 | hypothetical protein | gi\|45510532 |
| | | | Avar03000787 (pecB) | |
| | | | [Anabaena variabilis | |
| | | | ATCC 29413] | |
| 26.4 | 31044 | 32078 | cpcC | gi\|20141679 |
| | | | [Nostoc sp. PCC 7120] | |
| | | 32219 | hypothetical protein | gi\|45510539 |
| | | | Avar03000794 (rod | |
| | | | linker Mw 32000) | |
| | | | [Anabaena variabilis | |
| | | | ATCC 29413] | |
| | | 31295 | pecC | gi\|464511 |
| | | | [Nostoc sp. | |
| | | | PCC 7120] | |
| | | 31304 | hypothetical protein | gi\|45510534 |
| | | | Avar03000789 (pecC) | |
| | | | [Anabaena variabilis | |
| | | | ATCC 29413] | |
| | 30124 | 29333 | hypothetical protein | gi\|46135436 |
| | | | Avar03000801 (cpcG4) | |
| | | | [Anabaena variabilis | |
| | | | ATCC 29413] | |
| 26.8 | 26119 | 28637 | hypothetical protein | gi\|45510544 |
| | | | Avar03000799 (cpcG2) | |
| | | | [Anabaena variabilis | |
| | | | ATCC 29413] | |
| 27.8 | 10994 | 10986 | fdxH2: ferredoxin | gi\|1169673 |
| | | | vegetative | |
| | | | [Anabaena variabilis] | |
| 28.8 | 17714 | 17457 | cpcA | gi\|9957319 |
| | | | [Nostoc sp. PCC 7120] | |
| 30.0 | 19222 | 18332 | cpcB | gi\|38894 |
| | | | [Nostoc sp. PCC 7120] | |

Quantification of Phycocyanin:

To measure the molar concentration of pure PC we used its coefficient of molar extinction, ε, at 620 nm, which for the trimeric form, (αβ)₃, is equal to 770,000 M⁻¹cm⁻¹. This means that a solution of 1 mole of PC at 620 nm has an absorption value of 770,000. To measure the concentration of PC in the extract we use the coefficient of specific extinction E^{1%} at 620 nm of 70 1 g⁻¹ cm⁻¹. This means that a solution containing 1% of PC (that is 1 g/100 ml) at 620 nm absorbs 70. Based on these calculations, the average content of PC in the extract is equal to 80-100 mg/g DW (8-10% DW).

Purification of the PCB Chromophore:

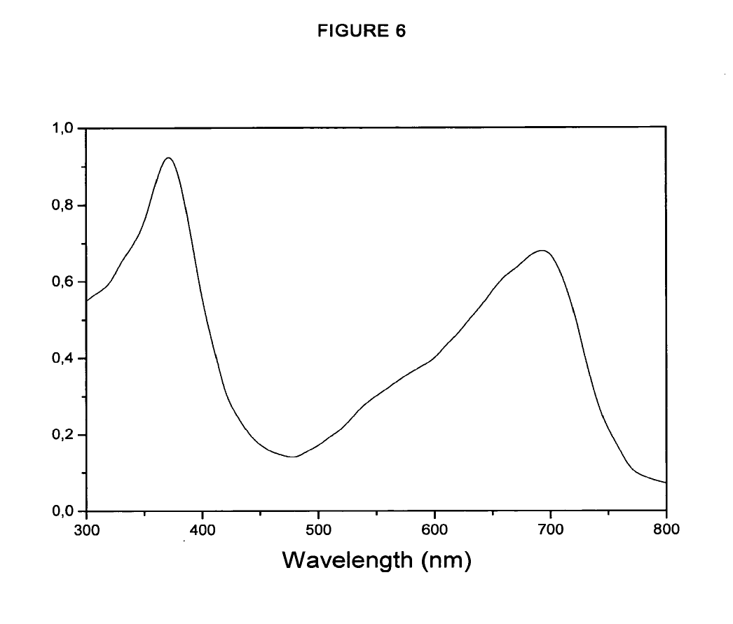

See FIG. 6.

suspend 500 mg of extract in 50 ml of distilled water;

centrifuge at 2500 rpm for 10 min at 4° C.;

decant the deep blue supernatant and precipitate the PC with 1% trichloroacetic acid;

incubate for 1 hr in the dark at 4° C., while agitating;

Centrifuge at 10,000 rpm for 30 min at 4° C.;

collect the pellet containing the PC and wash 3 times with methanol;

re-suspend the pellet in 10 ml of methanol containing 1 mg/ml of HgCl₂;

incubate for 20 hrs at 42° C. in darkness to release the PCB from PC;

centrifuge at 2,500 rpm for 10 min to separate the proteins;

collect the supernatant containing PCB and add β-mercaptoethanol (1 μl/ml) to precipitate the HgCl₂, and incubate at −20° C. for 24 hrs;

centrifuge at 10,000 rpm for 30 min at 4° C. to remove the white precipitate;

collect the supernatant and add 10 ml of methylene chloride/butanol (2:1, v/v);

wash the supernatant with 20 ml of distilled water & centrifuge at 3,000 rpm for 10 min.

remove the upper phase, harvest the lower part containing the PCB;

wash the PCB in 15 ml distilled water 3 times;

dry under nitrogen and store at −20° C.

Evaluation of the MAO-B Inhibition:

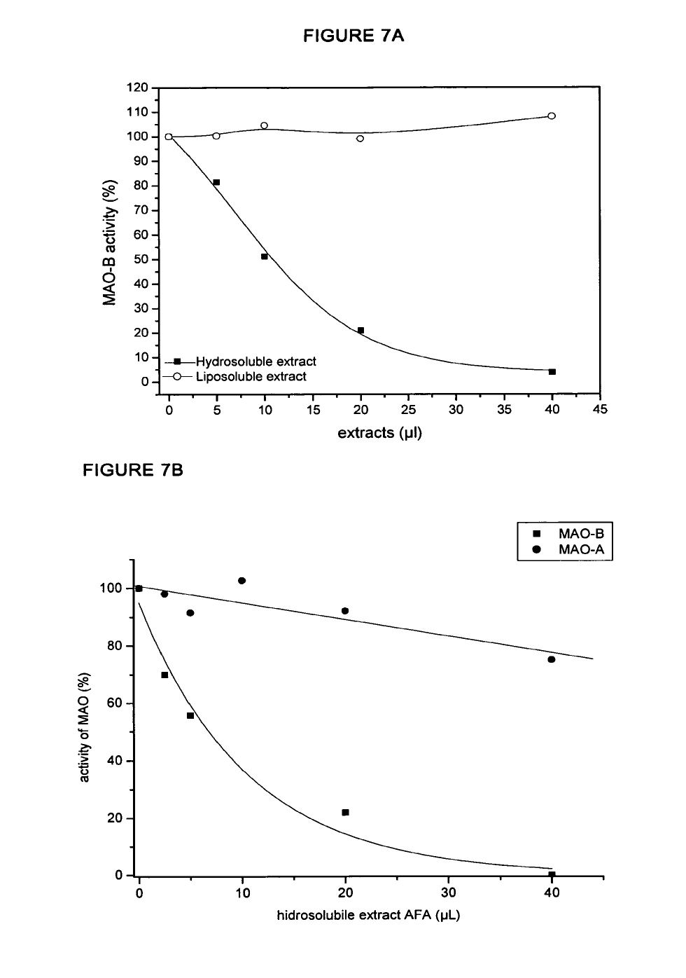

MAO-B inhibition is evaluated by AFA-Klamath Extract and by the extract's constitutive active principles: Phytochrome, Phycocyanin and MAAs. We have tested the MAO-B inhibitory activity of the Basic Extract using the specific substrate benzylamine (1 mM). The test was performed by a spectrophotometer at 30° C. at a wavelength of 250 nm, by pre-incubating MAO-B (2 μg/ml) with different concentrations of the water-soluble and lipid-soluble components of the Basic Extract, as produced by the steps a) to c) described above (initial concentration 10 mg/ml). The water-soluble, component-enriched extract was prepared by re-suspending the aqueous extract in water, then collecting the supernatant after centrifugation. The lipophilic component-enriched soluble extract has been obtained by re-suspending the extract in acetone; afterwards the supernatant has been dried, and the pellet has been re-suspended in DMSO, a solvent compatible with the dosage of MAO-B. As shown in FIG. 7A, the water-soluble fraction inhibits MAO-B in a dose-dependent manner, while the lipophilic fraction does not inhibit the enzyme. The water-soluble fraction of the AFA-Klamath Basic Extract is a potent selective MAO-B inhibitor, with an IC₅₀ of 6.9 μL. Its MAO-B selectivity is 4 (IC₅₀ MAO-B/IC₅₀ MAO-A>4.05) (FIG. 7B).

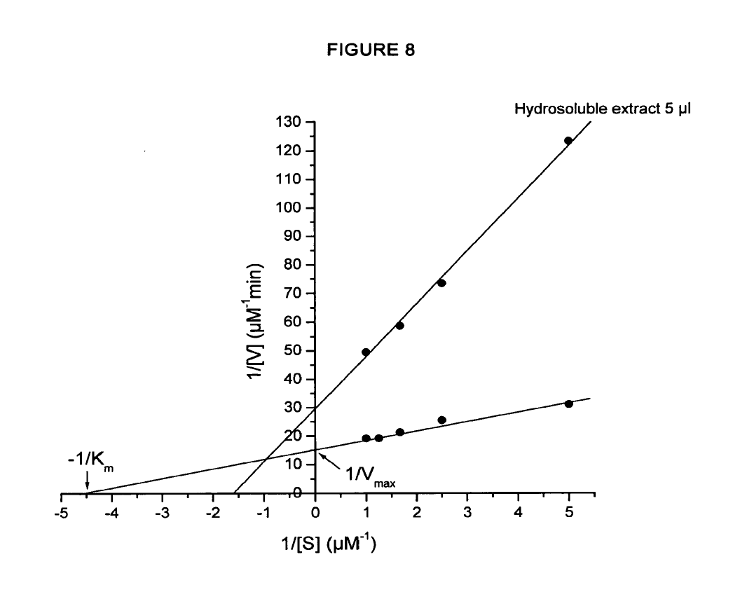

The Lineweaver-Burk plot in FIG. 8 shows that such inhibition is reversible and of a mixed type in relation to competition, with a decrease in the Vₘₐₓ and increase of the Michaelis-Menten Kₘ constant. Plotting the slope versus the concentration of the water-soluble fraction of the AFA-Klamath extract, a 1 μL inhibition constant Kᵢ is obtained. Compared to the water-soluble fraction of the Basic Extract, this low Kᵢ value indicates a high affinity for the MAO-B enzyme. The fact that the extract's inhibition is reversible means that it performs a physiological activity plausibly devoid of side effects. As to the mixed competition, it is very likely due to the complex nature of the extract, including different functional molecules, some competitive and others non-competitive. The main active components of the extract are the AFA-phytochrome (0.5% DW); phycocyanins (8-10% DW); and the MAAs or mycosporine-like amino acids (1.7-2.1% DW), which we have tested individually as MAO-B inhibitors.

MAO-B Inhibition by Phycocyanins:

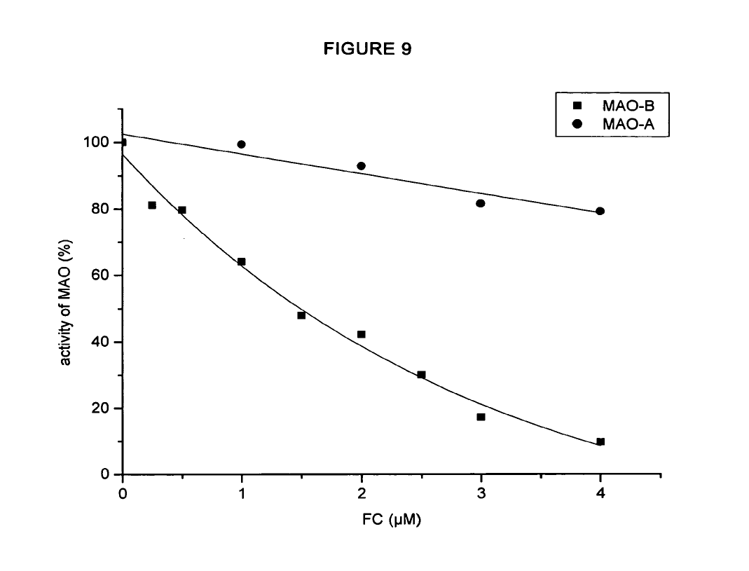

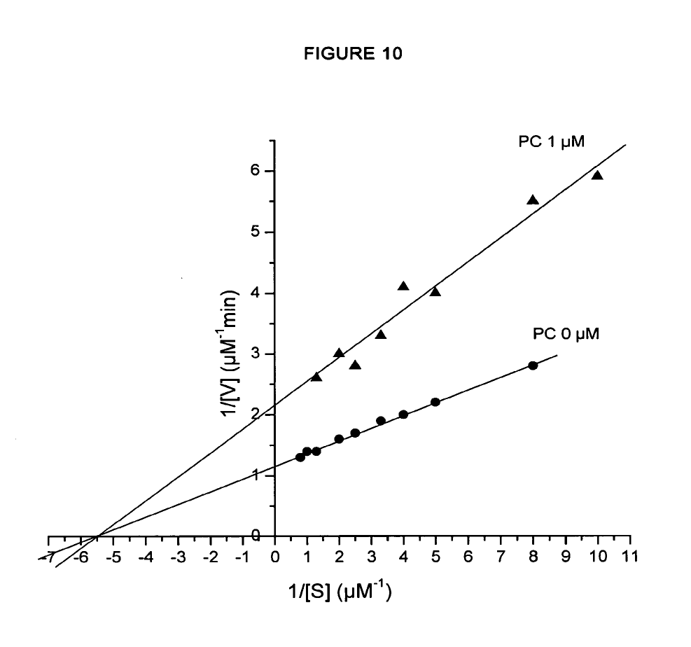

The test has been done using spectrophotometry at 30° C. at a wavelength of 250 nm, using benzylamine as a substrate, by preincubating MAO-B with various concentrations of purified PC from AFA-Klamath (0.5-4 μM). As shown in FIG. 9, AFA-PC causes a dose-dependent decrease of MAO-B activity, with an IC₅₀ of 1.44 μM. The MAO-B selectivity of AFA-PC is higher than 3.5 (IC₅₀MAO-B/IC₅₀MAO-A>3.5). The Lineweaver-Burk plot in FIG. 10 shows that, as with the extract, the inhibition is reversible and of a mixed type (competitive and non-competitive) with modification of both Vₘₐₓ and Kₘ. By plotting the slope versus the PC concentration, we obtain the value of the inhibition constant Kᵢ, which here is of 1.06 μM. The inhibition constant measures the affinity of the inhibitor for the enzyme: a high Kᵢ indicates a low affinity for the enzyme and vice-versa. In this instance, the low Kᵢ value indicates a high affinity of AFA-PC towards MAO-B.

MAO-B Inhibition by MAAs:

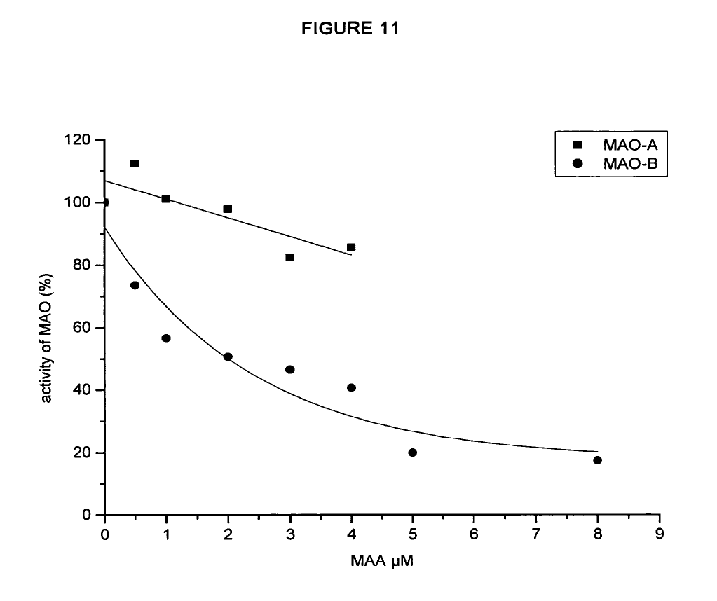

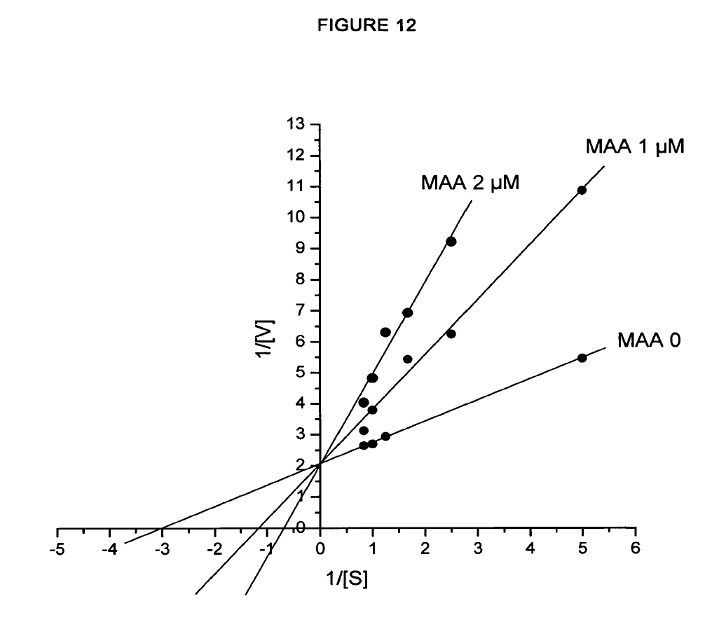

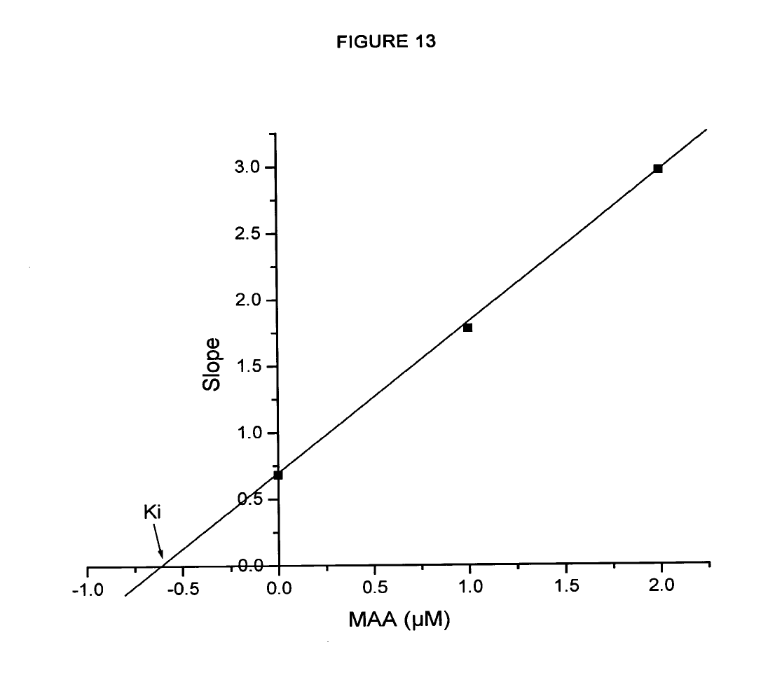

The activity of MAO-B on a benzylamine substrate has been evaluated in relation to increasing concentrations of MAAs (0.5-8 μM), previously purified from the Basic Extract with 20% methanol FIG. 11 shows the dose-dependent MAO-B inhibition by MAAs, with an IC₅₀ of 1.98 μM. The MAO-B selectivity of MAAs is higher than 2 (IC₅₀ MAO-B/IC₅₀ MAO-A>2.02). The Lineweaver-Burk plot (FIG. 12) shows that the inhibition is both reversible and competitive, with an increase of Kₘ but no variation of the Vₘₐₓ. This means that MAAs, thanks to their chemical structure, compete with the substrate for the link to the active site of the enzyme. Plotting the slope versus the concentration of MAAs (FIG. 13), we obtain the value of the inhibition constant Kᵢ, which is 0.585 μM, which demonstrates a very high degree of affinity for the enzyme.

MAO-B Inhibition by AFA-Phytochrome:

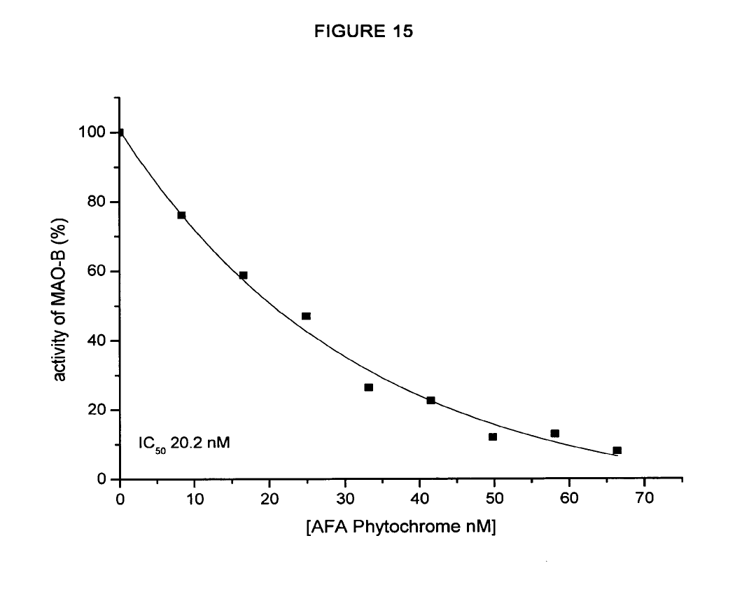

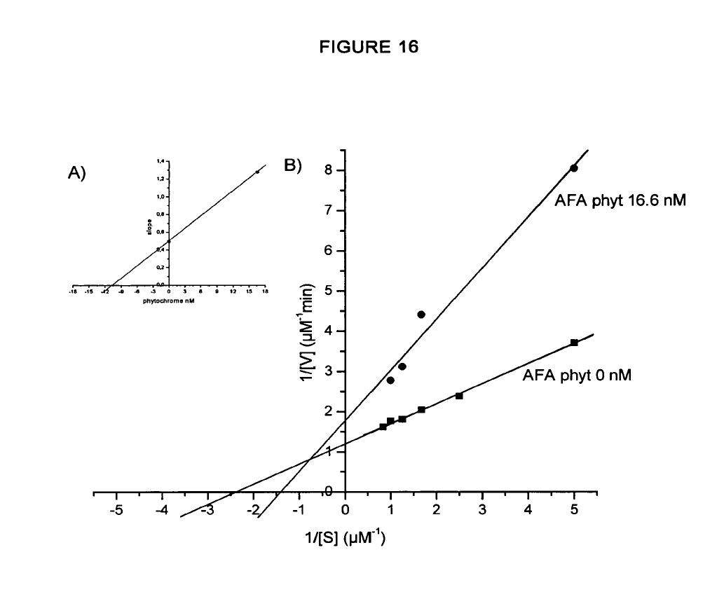

Testing was accomplished by spectrophotometry at 30° C. at a wavelength of 250 nm, using benzylamine as a substrate, by preincubating MAO-B with various concentrations of purified AFA-phytochrome (8.3-66.4 nM). As shown in FIG. 15, AFA-phytochrome causes a dose-dependent decrease of MAO-B activity, with an IC₅₀ as low as 20.2 nM. The Lineweaver-Burk plot in FIG. 16 shows that, as with the extract, the inhibition is reversible of a mixed type (competitive and non-competitive) with modification of both Vₘₐₓ and Kₘ. By plotting the slope versus the AFA-phytochrome concentration, we obtain the value of the inhibition constant Kᵢ, which here is 10.48 nM. The inhibition constant measures the affinity of the inhibitor for the enzyme: a high Kᵢ indicates a low affinity for the enzyme and vice-versa. In this instance, the extremely low Kᵢ value indicates a very high affinity of AFA-phytochrome towards MAO-B.

The competitive and reversible action of the MAAs makes these molecules very potent in the inhibition of MAO-B. Indeed, the competitive and reversible character of the MAO-B inhibition assures at the same time high efficacy and a physiological and side effects free activity. In this sense, the MAAs contained in the extract, also due to their molecular weight and consequent ability to easily cross the blood-brain barrier, constitute a decisive component, even in vivo, in order to generate the therapeutic effects derived from MAO-B inhibition. Even more than MAAs, the phytochrome has proven to be the most powerful MAO B inhibitor of all known substances to date. Its very high affinity for the MAO-B enzyme, and its effective inhibition at dosages of few nanomolars, make this molecule not only a perfect therapeutic agent on its own, but also the factor that seems to provide the most important contribution to the high neurological effectiveness of the AFA-Klamath extract(s). It should be added that some of the considerations relating to the MAAs and phytochromes could also be applied to the in vivo behavior of phycocyanins. We know that PC generate neuroprotective effects on the brain in vivo, and so that they are able to cross the blood-brain barrier. This means that they are also able to realize in vivo their MAO-B inhibitory activity in the brain. The molecular weight of the chromophore is indeed only 700, that is not much more than the molecular weight of the MAAs. The same holds true for the chromophore of the phytochrome, the phytochromobilin, structurally similar to phycocyanobilin.

In conclusion, the activity of MAO-B inhibition on the part of the extract and its active components, AFA-phytochrome, AFA-PC and MAAs, is extremely relevant, as both the molecules and the extract place themselves at the highest level of activity, equal or higher than the pharmacological substances, and greatly superior to any natural molecule tested, as shown in the following table:

TABLE 3
Comparative kinetics parameters (IC₅₀ & Kᵢ) of MAO B inhibition
of known synthetic and natural inhibitors.

| MAO-B Inhibitors | IC₅₀ | Kᵢ | Inhibition type |
|---|---|---|---|
| Deprenyl | 0.31 μM | 0.002 μM | Irreversible |
| Epicatechine | 58.9 μM | 21 μM | Mixed |
| Catechine | 88.6 μM | 74 μM | Mixed |
| Non Harman alkaloid | 6.47 μM | 112 μM | Mixed |
| Piperine | 91.3 μM | 79.9 μM | Competitive |
| Paeonol | 42.5 μM | 38.2 μM | Competitive |
| Emodin | 35.4 μM | 15.1 μM | Mixed |
| AFA phycocyanin | 1.44 μM | 1.06 μM | Mixed |
| AFA MAAs | 1.98 μM | 0.585 μM | Competitive |
| AFA phytochrome | 0.02 μM | 0.010 μM | Mixed |

As shown by the table, only phycocyanins and MAAs have an IC₅₀ slightly higher than 1 μM, thus very close to that of Deprenyl (0.31 μM), and tens of times lower than the IC₅₀ of the other molecules considered. AFA-phytochrome, on the other hand, has an IC₅₀ 15 times lower than that of Deprenyl. The same is true for the inhibition constant Kᵢ which measures the affinity of the inhibitor for the enzyme. AFA-phycocyanins have a Kᵢ of around 1 μM, like the non-Harman alkaloids of coffee and tobacco (but of course without any of the problems associated with those two substances). On the other hand, MAAs and the AFA-phytochrome are the only molecules, together with Deprenyl, to have a Kᵢ lower than 1 μM, and so a very high affinity for the MAO-B. In fact, AFA-phytochrome is the only natural molecule, besides Selegyline/Deprenyl, whose Kᵢ is in the order of a few nanomolar. And yet, there is an essential difference between Selegiline/Deprenyl and the molecules of the AFA-Klamath extract: the former is an irreversible inhibitor, thus characterized by potential side effects; whereas AFA-Klamath MAO-B inhibiting molecules are all reversible, characterized by a physiological activity devoid of the problems associated with synthetic molecules.

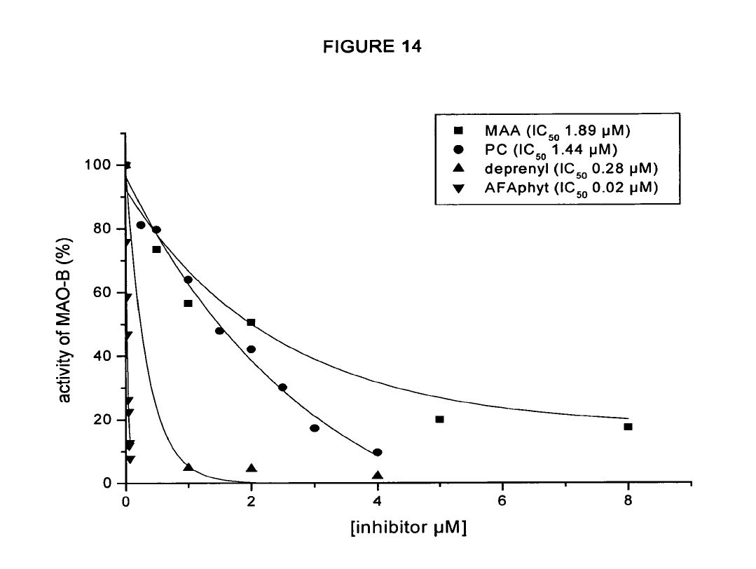

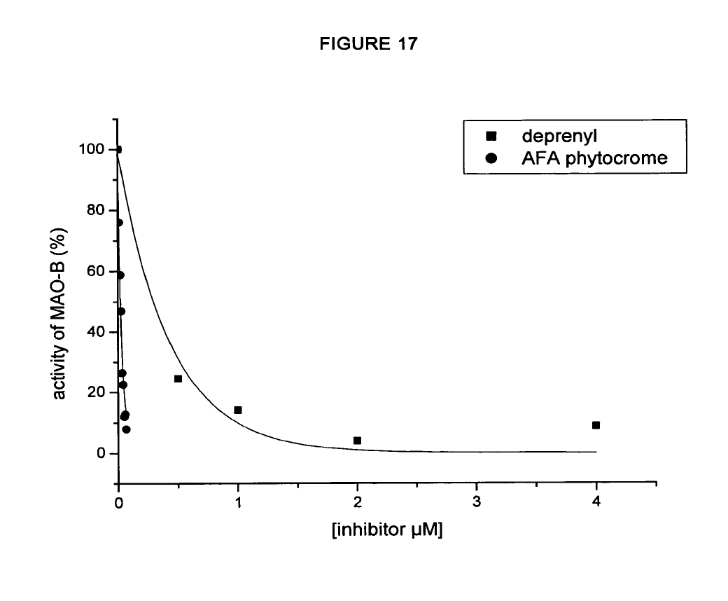

FIG. 14 shows graphically the MAO-B inhibitory activity of the three molecules of AFA-Klamath in relation to Deprenyl. Given the synergy of all three molecules in the Basic Extract (and other AFA-Klamath extracts), the overall MAO-B inhibitory activity of the Basic Extract results very high. Something that becomes particularly relevant considering also the high quantity of PEA present in it. If we compare the Basic Extract with Deprenyl on the base of its PC content, we obtain that the Basic Extract reaches the IC₅₀ at a PC dosage as low as 0.05 μM, which would indicate a potency 7.5 times higher than Deprenyl (and tens of times higher than the natural substances). This makes sense in light of the potency of the phytochrome contained in the Basic Extract: in fact 7.5 times is an average between the inhibitory potency of PC and MAAs, which is slightly lower than Deprenyl, and that of the phytochrome, which is 15 times higher (FIG. 17). This also shows that the higher potency of the extract relative to the purified AFA-PC is for the most part due to the phytochrome. Moreover, the extract still maintains the advantage of being a natural substance acting physiologically, whose MAO-B inhibition is reversible and mainly competitive, thus devoid of the side effects potentially associated with irreversible molecules such as Deprenyl and other synthetic substances.

The further advantage of the extract is its high content of phenylethylamine, a powerful dopaminergic neuro-modulator, which works in total synergy with other molecules. The synergistic activity can be thus summarized: Phenylethylamine (or “PEA”) has twofold dopaminergic activity, both as it stimulates the release of dopamine from the nigrostriatal tissue, and as it inhibits the post-synaptic reuptake of dopamine itself. Phytochrome, MAAs and phycocyanins, as powerful MAO-B inhibitors, also increase dopaminergic transmission insofar as a reduced activity MAO-B implies a longer life of neuroamines, including dopamine. Phytochrome, MAAs and phycocyanins, as MAO-B inhibitors, also prolong the life and activity of phenylethylamine, which is itself the object of the deamination activity of the MAO-B enzyme, with the consequent creation of a virtuous circle of further support to dopaminergic transmission and activity and to the more general neuro-modulation produced by PEA. Finally, the powerful antioxidant and anti-inflammatory activity of phycocyanins, together with their or their chromophore ability to cross the blood-brain barrier; as well as the extremely high antioxidant activity of the phytochrome and the less strong yet significant antioxidant activity of MAAs, generates a neuroprotection that shields the different active molecules and more generally the neurological virtuous cycle they create, from any oxidative and inflammatory damage.

Neuroprotection:

We have tested the neuroprotective properties of the AFA-Klamath extract, the specific AFA-PC and its chromophore PCB, as well as MAA's against the neurotoxic effect of glutamate. Glutamate is the main excitatory neurotransmitter in the mammalian central nervous system. However, over-stimulation of its NMDA subtype receptor in neurons triggers a massive intracellular accumulation of Ca²⁺, leading to cell death. In addition intra-mitochondrial Ca²⁺ accumulation, after NMDA receptor stimulation, causes transient increases in free cytosolic Ca²⁺ activate the neuronal isoform of nitric oxide synthase (NOS), an enzyme that forms nitric oxide (NO.) or, mainly in primary neurons, its superoxide (O2.-) reaction product, peroxynitrite (ONOO.). The exposure of neurons to glutamate was carried according to a slightly modified method: culture medium was removed and neurons were washed once with pre-warmed 37° C. buffered Hanks' solution (5.26 mM KCl, 0.43 mM KH₂H₂PO₄, 132.4 mM NaCl, 4.09 mM NaHCO₃, 0.33 mM Na₂HPO₄, 20 mM glucose, 2 mM CaCl₂, and 20 mM HEPES at pH 7.4) and pre-incubated in the absence or presence of several concentrations of AFA-Klamath extract (1-50 nM), PC (10-1000 nM), PCB (10-1,000 nM) and MAA (1-10 μM) in pre-warmed 37° C. buffered Hanks' solution. After 30 min of pre-incubation, L-glutamate was added from concentrated solutions to the final concentration indicated 100 μM plus 10 μM glycine. Neurons were incubated at 37° C. for 15 min, the buffer was aspirated, replaced with DMEM and the cells were incubated at 37° C. for another 24 hrs in the absence of effectors.

Apoptosis was assessed by staining the nuclei of cells with DAPI, a membrane-permeable fluorescent dye that binds DNA and allows quantification of apoptotic neurons, i.e., neurons displaying fragmented or condensed nuclei. Briefly, 24 hrs after glutamate exposure, neuronal cultures were washed with warm (37° C.) PBS and fixed with 4% (wt/vol) paraformaldehyde in PBS for 30 min at room temperature. After being washed with PBS, cells were exposed to 3 μM DAPI for 10 min at room temperature in the dark and were then washed twice with PBS. Cells were scored for chromatin condensation by fluorescence microscopy, using a fluorescein filter (330-380 excitation; 30× magnification). Total and apoptotic nuclei were counted. In all cases, approximately 600-1,000 cells were counted per well by an operator blind to the protocol design. Measurements from individual cultures were performed in duplicate and results are expressed as the mean S.E.M. values for the number of culture preparations indicated. Statistical analysis of the results was determined by Kruskal-Wallis test followed by the least significant difference multiple range test. In all cases, p-0.05 was considered significant.

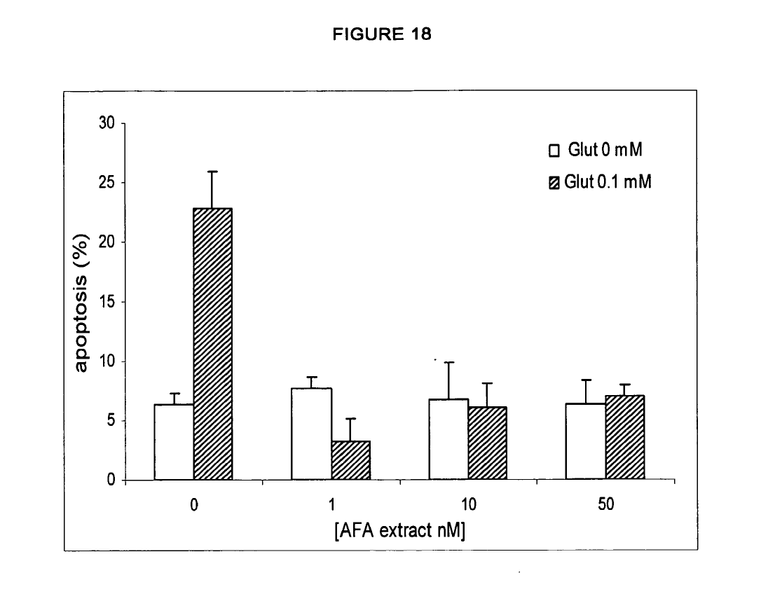

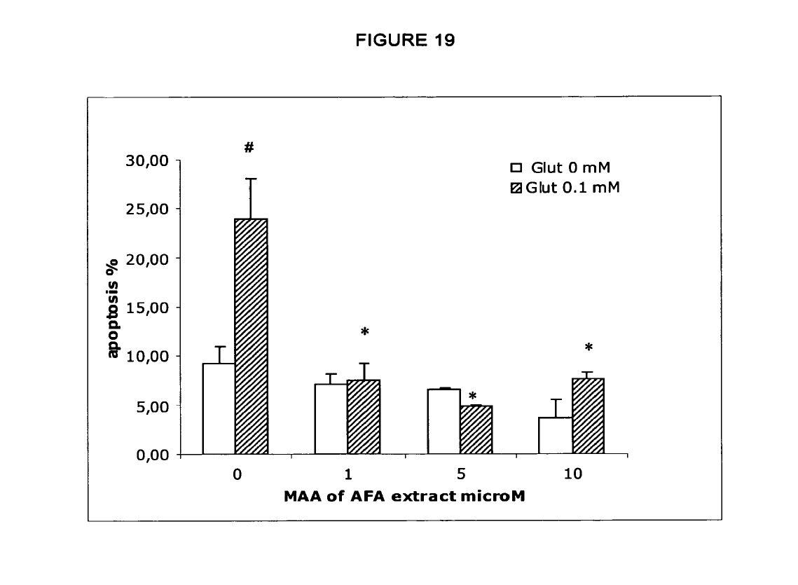

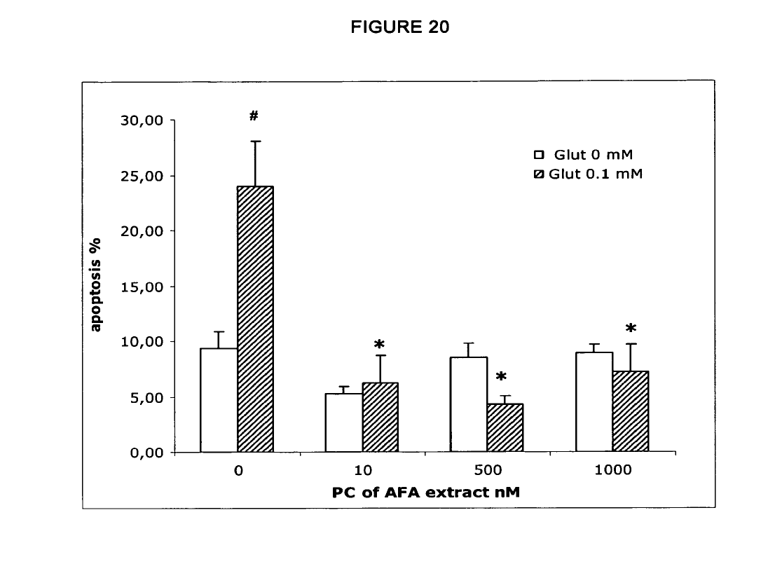

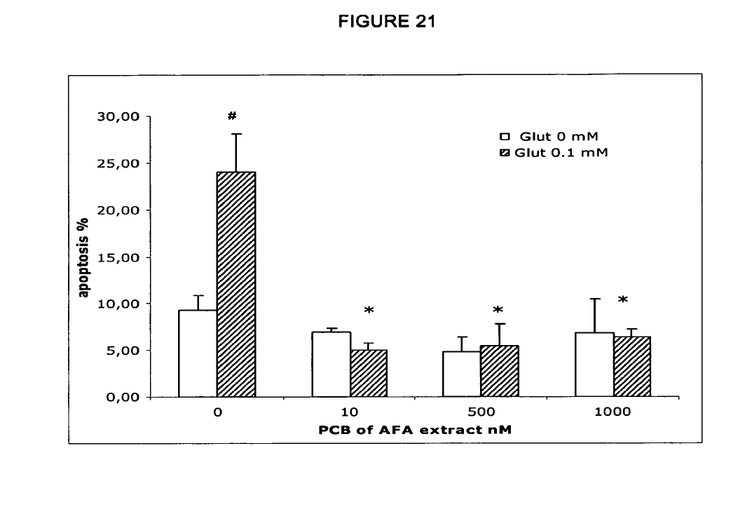

Through this glutamate damage test we have shown for the first time the neuroprotective ability of AFA-Klamath Basic Extract, AFA-PC, its PCB and MAAs. As shown by FIG. 18, the addition of glutamate to the cultured neuron cells has increased the level of apoptosis to a percentage of 22.9%+/−0.3 n=4 (p<0.05); while the simultaneous addition of the AFA-Klamath Basic Extract has generated a very high protection against glutamate toxicity, lowering the level of apoptosis below the control level of 6.3%+/−0.1 (p>0.05) already with as low an amount of extract as 1 nM (results are means+/−SEM from 3 to 8 different cell cultures. FIG. 19 shows: # significantly different when compared with control group (p<0.05); * significantly different when compared with the glutamate control (p<0.05). As to the protection afforded by MAAs, they also lower the level of apoptosis below the control level, with the higher dosage of 1 μM. Results are means+/−SEM from 3 to 8 different cell cultures. FIGS. 20 & 21 show: # significantly different when compared with control group (p<0.05); * significantly different when compared with the glutamate control (p<0.05). Regarding AFA-PC and PCB, we see that their inhibition of apoptosis is very similar their addition to the cell culture lowers the degree of apoptosis below the control with a dosage of approximately 10 nM (FIGS. 20 and 21)—results are means+/−SEM from 3 to 8 different cell cultures.

The degree of inhibition of AFA-PC is approximately equal to that of PCB. This is somewhat surprising, given that the PCB, supposedly its most active principle, once purified and thus more concentrated, should be significantly stronger than the whole molecule of which is the active component. The fact that it has practically the same potency means that in the whole PC there are other factors that may actually be even more potent than the PCB itself. We know that the whole PC is composed, besides C-PC and its PCB chromophore, of PEC, which includes as its chromophores both PCB and PVB (phycoviolobilin). Therefore, we can here assume that the factor that creates a significant difference in potency between the purified PCB and the whole PC is precisely the PEC component, particularly its PVB chromophore, which is assumed to be a very strong antioxidant.

In terms of neuro-protection, MAAs seem to play a role, but significantly less than PC and PCB. However, the most powerful neuro-protectant is clearly the whole AFA-Klamath extract, which is able to completely inhibit cell apoptosis at just 1 nM. This is 10 times the potency of PC and PCB. This can certainly be explained with the synergy of many different antioxidant factors present in the whole AFA-Klamath extract; yet, since we have seen above that the AFA-phytochrome is possibly the most powerful antioxidant to date, being able to almost completely inhibit MDA (a late by-product of lipid-peroxidation) formation with just 16 nanomolar, it is very likely that AFA-Phytochrome is the more important factor in explaining the higher potency of the Basic Extract. We can thus conclude that AFA-Phytochromes, as well as any and all phytochromes, are important neuroprotective agents.

While the above description contains many specifics, these should not be construed as limitations on the scope of the invention, but rather as exemplifications of one or another embodiment thereof. Many other variations are possible, which would be obvious to one skilled in the art. Accordingly, the scope of the invention should be determined by the scope of the appended claims and their equivalents, and not just by the embodiments.

What is claimed is:

1. A body treating preparation comprising at least one isolated component of microalga AFA-Klamath, wherein the isolated component is selected from the group AFA derived components consisting of: C-Phycocyanin (C-PC), Phycoerythrocyanin (PEC), a C-Phycocyanin/Phycoerythrocyanin complex (C-PC/PEC), Chromophore Phycocyanobilin (PCB), Chromophore Phycoviolobilin (PVB), an AFA-Phytochrome, Phenylethylamine (PEA), and Mycosporine-like Amino Acids (MAAs); and said preparation further adapted to be physiologically suitable for treating a human body.

2. The preparation according to claim 1, wherein the isolated component is a Mycosporine-like Amino Acid selected from Shinorine and Porphyra-334.

3. The preparation according to claim 1, wherein the isolated component comprises at least one component of a group of components consisting of: the C-Phycocyanin/Phycoerythrocyanin complex component, the C-Phycocyanin component, and the Phycoerythrocyanin component.

4. The preparation according to claim 1, wherein the isolated component is the AFA-Phytochrome component.

5. The preparation according to claim 1, wherein the isolated component comprises at least one component of a group of components consisting of: Mycosporine-like Amino Acids, Phycocyanin, Phycoerythrocyanin, and Phytochrome.

6. The preparation according to claim 5 additionally containing the isolated component C-Phenylethylamine.

7. The preparation according to claim 1, wherein said preparation further comprises a pharmaceutically acceptable excipient suitable for treating a human body.

8. The preparation according to claim 1, wherein the isolated components comprise at least one component of a group of components consisting of: AFA-phycocyanin and Chromophore Phycocyanobilin.

9. The preparation according to claim 1, wherein the isolated components have a molecular weight cut-off of less than about 30,000 to 40,000 Dalton.

10. The preparation according to claim 1, wherein a daily treatment of the body treating preparation comprises Phenylethylamine at a concentration of about 1 to 10 mg.

11. The preparation according to claim 1, wherein a daily treatment of the body treating preparation comprises Phenylethylamine at a concentration of about 5 to 20 mg.

12. The preparation according to claim 1, wherein a daily treatment of the body treating preparation comprises: Phenylethylamine at about 5 to 30 mg, the AFA-Phytochrome at about 0.8 to 10 mg, the Mycosporine-like Amino Acids at about 10 to 100 mg, and the C-Phycocyanin and Phycoerythrocyanin combined at about 50 to 1,000 mg.

13. The preparation according to claim 1, wherein a daily treatment of the body treating preparation comprises: the Phenylethylamine at about 0.1 to 100 mg, the AFA-Phytochrome at about 0.1 to 1,000 mg, the Mycosporine-like Amino Acids at about 0.1 to 1,000 mg, and the C-Phycocyanin and Phycoerythrocyanin combined at about 1 to 2,500 mg.

14. The preparation according to claim 1, wherein the preparation selectively inhibits monoamineoxidase B (MAO-B) with an IC₅₀ of 6.9 pL and a selectivity of 4.

15. The preparation according to claim 1, wherein the AFA-phytochrome is at about 0.5% Dry Weight, the Mycosporine-like Amino Acids are at about 1.7 to 2.1% Dry Weight, and the C-Phycocyanin and Phycoerythrocyanin combined are at about 8 to 10% Dry Weight.
